SEQUENCE LISTING (12 sequences)

SEQ ID NO: 1 - protein, 3 aa, Artificial
  RGD peptide
RGD

SEQ ID NO: 2 - protein, 6 aa, Artificial
  RGD peptide
GRGDSP

SEQ ID NO: 3 - protein, 5 aa, Artificial
  RGD peptide
RGDFV

SEQ ID NO: 4 - DNA, 22 bp, Artificial
  rabbit AGGR-F
gatctaccgctgtgaggtgatg

SEQ ID NO: 5 - DNA, 20 bp, Artificial
  rabbit AGGR-R
cctttcaccacgacctccaa

SEQ ID NO: 6 - DNA, 25 bp, Artificial
  TaqMan&reg; probe
acggccttgaggacagcgaggctac

SEQ ID NO: 7 - DNA, 18 bp, Artificial
  rabbit COL2-F
cccccgctctccaagaga

SEQ ID NO: 8 - DNA, 28 bp, Artificial
  rabbit COL2-R
gccaggaagacaataaataaatagaaca

SEQ ID NO: 9 - DNA, 29 bp, Artificial
  TaqMan probe
tgaactgggcagactgcaaaacaaaagct

SEQ ID NO: 10 - DNA, 19 bp, Artificial
  rabbit SOX9-F
agtacccgcacctgcacaa

SEQ ID NO: 11 - DNA, 20 bp, Artificial
  rabbit SOX9-R
cgcttctcgctctcgttcag

SEQ ID NO: 12 - DNA, 24 bp, Artificial
  TaqMan probe
agctcagcaagaccctcgggaagc

(19) United States
(12) Patent Application Publication
(10) Pub. No.: US 2012/0122791 A1
(43) Pub. Date: May 17, 2012

(54) SUBSTRATE FOR CARTILAGE CULTIVATION USING ARTIFICIAL COLLAGEN, AND METHOD FOR CARTILAGE REGENERATION TREATMENT USING THE SUBSTRATE

(75) Inventors: Masahiko Suzuki, Chiba-shi (JP); Koichi Ueno, Chiba-shi (JP); Nanami Muto, Chiba-shi (JP)

(73) Assignees: JNC CORPORATION, Tokyo (JP); Masahiko SUZUKI, Chiba-shi (JP)

(21) Appl. No.: 13/318,312

(22) PCT Filed: Feb. 2, 2010
(86) PCT No.: PCT/JP2010/001163

§ 371 Date: Jan. 27, 2012

(30) Foreign Application Priority Data
May 1, 2009 (JP) 2009-112217

(51) Int. Cl. A61K 38/02; A61P 19/02; A61P 19/04; C07K 14/47

(52) U.S. Cl. 514/17.1; 530/350

(57) ABSTRACT

Provided are a substrate for cartilage cultivation, which exhibits high safety and has a growth promoting effect on cartilage cells, and a method for cartilage regeneration treatment. The substrate for cartilage cultivation containing artificial collagen, in particular, an artificial collagen aqueous solution has a growth promoting effect on cartilage cells. A cartilage regeneration effect is obtained by the intraarticular injection of the substrate in and around a cartilage defect site.

This application is a U.S. National Phase under 35 U.S.C. §371 of International Application PCT/JP2010/001163, filed on Feb. 23 2010, which claims priority upon Japanese application 2009-112217, filed on May 1, 2009; the contents of which are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF THE INVENTION

1. Field of the Invention

The present invention relates to a substrate for cartilage cultivation using artificial collagen, and a method for cartilage regeneration treatment using the substrate. It should be noted that the present application claims a priority based on Japanese Patent Application No. 2009-112217, which is incorporated by reference herein.

2. Description of the Related Art

Collagen is a fibrous protein, serves as a major component of the skin or the bone, and is said to account for about 30% (W/W) of the total protein in mammals. Further, in a general collagen molecule, three collagen polypeptide chains forms a rope-like superhelical structure called a triple-helical structure. In addition, the contents of proline (Pro), glycine (Gly) and hydroxyproline (Hyp) are particularly high in collagen, and both amino acid residues are important for forming a stable triple-helical structure.

For methods of preparing and utilizing collagen of biological origin, there are several exemplifications; a method of grafting an untreated or lyophilized porcine or bovine skin tissue to a skin site damaged by a burn or the like, a method of removing cell components by an enzyme treatment or the like before use, and a method of solubilizing collagen by an acidic solution or an enzyme treatment, and reconstructing the collagen to a desired form before use.

Meanwhile, bovine spongiform encephalopathy is caused by an infectious protein called a prion, and the infectious protein is said to be one of causes of Creutzfeldt-Jakob disease infection in a human. The prion is a protein and is hardly inactivated by general sterilization and disinfection methods. It has also been pointed out that a prion infection occurs across species. Further, it is not deniable that collagen of biological origin may have been contaminated with a virus.

In general, collagen of bovine or porcine origin is often used as a raw material for medical devices, pharmaceuticals, or cosmetics. Therefore, there always exists a risk of infection with pathogens such as a prion which cannot be removed by general sterilization and disinfection methods.

In addition, collagen of biological origin is a xenogeneic protein for patients who will undergo grafting, and hence, there has also been a problem associated with an immune rejection reaction.

Meanwhile, a technique called mosaicplasty involving samples of several columns of cartilage tissue in an unloaded site, and regrafting the cartilage tissue to an affected part has been mainly employed for treatment of a cartilage defect site. However, the use of an autologous tissue imposes a large burden on patients, and further, the sampling amount of the tissue is restricted.

Further, Japanese Patent Application Laid-open No. 2004-194944 (PTL1) reports a substrate for cartilage cultivation using collagen and its manufacturing method. However, the patent document uses collagen of biological origin. In addition, Japanese Patent Application Laid-open No. 2003-180815 (PTL2) reports a resorbable extracellular matrix containing collagen I and collagen II for cartilage reconstruction. However, the patent document uses collagen of biological origin similarly to the above-mentioned document.

Japanese Patent Application Laid-open No. 2003-321500 (PTL3) and Japanese Patent Application Laid-open No. 2005-58499 (PTL4) each report artificial collagen. However, Japanese Patent Application Laid-open No. 2003-321500 does not disclose utilization of artificial collagen as a scaffold for cartilage cells, and a growth promoting effect on cartilage cells. Further, Japanese Patent Application Laid-open No. 2005-58499 does not disclose a growth promoting effect of artificial collagen, in particular, an artificial collagen aqueous solution on cartilage cells.

### Citation List

### Patent Literature

[PTL1] Japanese Patent Application Laid-open No. 2004-194944

[PTL2] Japanese Patent Application Laid-open No. 2003-180815

[PTL3] Japanese Patent Application Laid-open No. 2003-321500

[PTL4] Japanese Patent Application Laid-open No. 2005-58499

### SUMMARY OF THE INVENTION

### Technical Problem

The present invention has been made in order to satisfy the above-mentioned demand, and therefore has an object to provide a substrate for cartilage cultivation which exhibits high safety and has a growth promoting effect on cartilage cells, and a method for cartilage regeneration treatment using the substrate.

### Solution to Problem

The inventors of the present invention have intensively studied in order to achieve the above-mentioned object. As a result, the inventors have found that a substrate for cartilage cultivation containing an artificial collagen, in particular, an artificial collagen aqueous solution satisfies the above-mentioned demand. Thus, the present invention has been completed.

That is, the present invention provides the following:

1. A substrate for cartilage cultivation, including an artificial collagen.

2. A substrate for cartilage cultivation according to the item 1, in which the artificial collagen includes a solution having a concentration of 0.001 to 6.00% (W/V).

3. A substrate for cartilage cultivation according to the item 2, in which the artificial collagen includes an aqueous solution having a concentration of 0.001 to 6.00% (W/V).

4. A substrate for cartilage cultivation according to any one of the items 1 to 3, in which the artificial collagen includes a polypeptide formed of peptide units represented by the following (1) to (3):

[—(OC—(CH₂)ₘ—CO)ₚ-(Pro-Y-Gly)ₙ-]ₐ; (1)

[—(OC—(CH₂)ₘ—CO)_{q}—(Z)ᵣ—]_{b}; (2)

and

[—HN—R—NH—]_{c}, (3)

where: m represents an integer of 1 to 18, p and q are identical to or different from each other and each represent 0 or 1, Y represents Pro or Hyp, and n represents an integer of 1 to 20; Z represents a peptide chain formed of 1 to 10 amino acid residues, r represents an integer of 1 to 20, and R represents a linear or branched alkylene group; and a ratio of a to b is a/b=100/0 to 30/70 (molar ratio), c=a is satisfied if p=1 and q=0, c=b is satisfied if p=0 and q=1, c=a+b is satisfied if p=1 and q=1, and c=0 is satisfied if p=0 and q=0.

5. A substrate for cartilage cultivation according to the item 4, in which m represents an integer of 2 to 12, n represents an integer of 2 to 15, Z represents a peptide chain formed of at least one kind of amino acid residue or peptide residue selected from Gly, Sar, Ser, Glu, Asp, Lys, His, Ala, Val, Leu, Arg, Pro, Tyr, and Ile, r represents an integer of 1 to 10, and R represents a C₂ to C₁₂ alkylene group.

6. A substrate for cartilage cultivation according to any one of the items 1 to 5, further including sodium hyaluronate.

7. A substrate for cartilage cultivation according to any one of the items 1 to 6, further including an RGD peptide.

8. A substrate for cartilage cultivation according to any one of the items 1 to 7, including a scaffold material for cartilage cells.

9. A substrate for cartilage cultivation according to any one of the items 1 to 7, including a material for differentiation and growth of cartilage cells.

10. A joint function improving agent, including sodium hyaluronate and artificial collagen.

11. A joint function improving agent according to the item 10, including the artificial collagen as an aqueous solution having a concentration of 0.001 to 6.00% (W/V).

12. A joint function improving agent according to the item 10 or 11, in which the artificial collagen includes a polypeptide formed of peptide units represented by the following (1) to (3):

[—(OC—(CH₂)ₘ—CO)ₚ-(Pro-Y-Gly)ₙ-]ₐ; (1)

[—(OC—(CH₂)ₘ—CO)_{q}—(Z)ᵣ—]_{b}; (2)

and

[—HN—R—NH—]_{c}, (3)

where: m represents an integer of 1 to 18, p and q are identical to or different from each other and each represent 0 or 1, Y represents Pro or Hyp, and n represents an integer of 1 to 20; Z represents a peptide chain formed of 1 to 10 amino acid residues, r represents an integer of 1 to 20, and R represents a linear or branched alkylene group; and a ratio of a to b is a/b=100/0 to 30/70 (molar ratio), c=a is satisfied if p=1 and q=0, c=b is satisfied if p=0 and q=1, c=a+b is satisfied if p=1 and q=1, and c=0 is satisfied if p=0 and q=0.

13. A joint function improving agent according to the item 12, in which m represents an integer of 2 to 12, n represents an integer of 2 to 15, Z represents a peptide chain formed of at least one kind of amino acid residue or peptide residue selected from Gly, Sar, Ser, Glu, Asp, Lys, His, Ala, Val, Leu, Arg, Pro, Tyr, and Ile, r represents an integer of 1 to 10, and R represents a C₂ to C₁₂ alkylene group.

14. Use of the substrate for cartilage cultivation according to any one of items 1 to 9 for cartilage regeneration treatment including the intra-articular injecting of the substrate for cartilage cultivation in and around a cartilage defect site or a cartilage disorder site of a patient.

15. Use of the substrate for cartilage cultivation according to any one of items 1 to 9 for cartilage regeneration-promotion treatment including the intra-articular injecting of the substrate for cartilage cultivation in and around a cartilage transplantation of a patient after the cartilage transplantation.

16. Use of the substrate for cartilage cultivation according to any one of items 1 to 9 for treatment of protecting joint function including intra-articularly injecting the substrate for cartilage cultivation in and around a cartilage defect site or a cartilage disorder site of a patient suffering with joint damage.

17. Use of the substrate for cartilage cultivation according to item 16, in which the joint damage is selected from any one of the following:

(1) joint damage due to osteoarthritis;

(2) joint damage due to injury;

(3) joint damage due to sports;

(4) joint damage due to rheumatoid arthritis; and

(5) joint damage due to connective tissue disorder such as systemic lupus erythematosus.

18. A method for cartilage regeneration treatment, including the intra-articular injecting of the substrate for cartilage cultivation according to any one of items 1 to 9 in and around a cartilage defect site or a cartilage disorder site of a patient.

19. A method for cartilage regeneration-promotion treatment, including the intra-articular injecting of the substrate for cartilage cultivation according to any one of items 1 to 9 in and around a site of a cartilage transplantation of a patient after the cartilage transplantation.

20. A method for treatment of protecting joint function, including the intra-articular injecting of the substrate for cartilage cultivation according to any one of items 1 to 9 in and around a cartilage defect site or a cartilage disorder site of a patient suffering with joint damage.

21. A method for treatment of protecting joint function according to the item 20, in which the joint damage is selected from any one of the following:

(1) joint damage due to osteoarthritis;

(2) joint damage due to injury;

(3) joint damage due to injury;

(4) joint damage due to rheumatoid arthritis; and

(5) joint damage due to connective tissue disorder such as systemic lupus erythematosus.

### Advantageous Effects of Invention

The present invention can provide a substrate for cartilage cultivation which exhibits high safety and has a growth promoting effect on cartilage cells, and a method for cartilage regeneration treatment using the substrate.

### BRIEF DESCRIPTION OF THE DRAWINGS

In the accompanying drawings:

FIG. 1 is photographs showing toluidine blue stained specimens of artificial collagen and bovine type 2 collagen;

FIG. 2 is a photograph showing a toluidine blue stained specimen of artificial collagen;

FIG. 3 is a graph illustrating the measurement results of a proteoglycan amount;

FIG. 4 is a graph illustrating the measurement results of a DNA amount;

FIG. 5 is a graph illustrating the results of the ratio of the proteoglycan amount and the DNA amount;

FIG. 6 is a graph illustrating the measurement results of an aggrecan gene expression amount;

FIG. 7 is a graph illustrating the measurement results of a type 2 collagen gene expression amount;

FIG. 8 is a graph illustrating the measurement results of a sox9 gene expression amount;

FIG. 9 is a graph illustrating the measurement results of a proteoglycan production ability of cartilage cells by addition of an artificial collagen aqueous solution;

FIG. 10 is a graph illustrating the measurement results of a type 2 collagen mRNA amount in cartilage cells by addition of an artificial collagen aqueous solution;

FIG. 11 is a graph illustrating the measurement results of an aggrecan mRNA amount in cartilage cells by addition of an artificial collagen aqueous solution (abscissa axis indicating the aggrecan mRNA amount);

FIG. 12 is photographs showing images of knee joints of the respective groups, and a graph illustrating the results with a modified ICRS score;

FIG. 13 is photographs showing images in which knee joints of the respective groups have been stained with Safranin-0 (Rosenburg, J Bone Joint Surg, 53A:69-82, 1971), and a graph illustrating the results of a Safranin-0-stained area ratio;

FIG. 14 is photographs showing images in which knee joints of the respective groups have been immunostained with a type 2 collagen antibody, and a graph illustrating the results of a type 2 collagen antibody-immunostained area ratio; and

FIG. 15 is photographs showing images of the piece of cartilage after organ cultivation (upper left: stained with safranin-o, left below: enlarged view, lower right: enlarged view with fluorescent label).

### DESCRIPTION OF EMBODIMENTS

Hereinafter, the present invention is described in detail.

### Artificial Collagen

Artificial collagen of the present invention means that it is not collagen of biological origin. For example, a polypeptide as described below is used as the artificial collagen of the present invention.

A polypeptide as the artificial collagen of the present invention is formed of peptide units represented by the following formulae (1) to (3):

[—(OC—(CH₂)ₘ—CO)ₚ-(Pro-Y-Gly)ₙ-]ₐ; (1)

[—(OC—(CH₂)ₘ—CO)_{q}—(Z)ᵣ—]_{b}; (2)

and

[—HN—R—NH—]_{c}, (3)

where: m represents an integer of 1 to 18, p and q are identical to or different from each other and each represent 0 or 1, Y represents Pro or Hyp, and n represents an integer of 1 to 20; Z represents a peptide chain formed of 1 to 10 amino acid residues, r represents an integer of 1 to 20, and R represents a linear or branched alkylene group; and a ratio of a to b is a/b=100/0 to 30/70 (molar ratio), c=a is satisfied if p=1 and q=0, c=b is satisfied if p=0 and q=1, c=a+b is satisfied if p=1 and q=1, and c=0 is satisfied if p=0 and q=0. More specifically, in a polypeptide formed of units represented by the formulae (1) to (3), m represents an integer of 2 to 12, n represents an integer of 2 to 15, and Z represents a peptide chain formed of at least one kind of amino acid residue or peptide residue selected from Gly, Sar, Ser, Glu, Asp, Lys, His, Ala, Val, Leu, Arg, Pro, Tyr, and Ile in general. In addition, in the formulae, r represents an integer of 1 to 10, and R represents a C₂ to C₁₂ alkylene group in general.

Further, a polypeptide of the present invention may be formed of the following repetitive unit (i), (ii), or (iii):

(i) a polypeptide formed of a repetitive unit containing a peptide unit [-(Pro-Y-Gly)ₙ-]ₐ (where Y and n have the same meanings as above) and a peptide unit [—(Z)ᵣ—]_{b} (where Z and r have the same meanings as above) at a ratio of a/b=100/0 to 40/60 (molar ratio);

(ii) a polypeptide formed of a repetitive unit containing a peptide unit [—(OC—(CH₂)ₘ—CO)-(Pro-Y-Gly)ₙ-]ₐ (where m, n, and Y have the same meanings as above) and a unit [—HN—R—NH—]_{c} (where R has the same meaning as above) at a ratio of substantially a/c=1/1 (molar ratio); and

(iii) a polypeptide formed of a repetitive unit containing a peptide unit [—(OC—(CH₂)ₘ—CO)-(Pro-Y-Gly)ₙ-]ₐ (where m, n, and Y have the same meanings as above), a peptide unit [—(OC—(CH₂)ₘ—CO)—(Z)ᵣ—]_{b} (where m, r, and Z have the same meanings as above), and a unit [—HN—R—NH—]_{c} (where R has the same meaning as above) at a ratio of a/b=100/0 to 40/60 (molar ratio) and substantially at a ratio of (a+b)/c=1/1 (molar ratio).

It should be noted that the linear or branched alkylene group represented by R as described above may be any alkylene group as long as physical and biological properties of the polypeptide are not impaired. Examples of the alkylene group include a C₁ to C₁₈ alkylene group such as methylene, ethylene, propylene, trimethylene, and tetramethylene. The alkylene group R may also be a linear methylene chain (CH₂)ₛ (s represents an integer of 1 to 18). R is preferably a C₂ to C₁₂ alkylene group (more preferably a C₂ to C₁₀ alkylene group, or particularly preferably a C₂ to C₆ alkylene group). It should be noted that details and production methods of those artificial collagens are described in Japanese Patent Application Laid-open No. 2003-321500.

In addition, preferably, artificial collagen (INCI name: Poly(Tripeptide-6), CAS. No: 60961-94-6: http://www/phg.co.jp/research/collagen.html) sold by PHG Corporation is preferably used as the artificial collagen of the present invention.

The desired shape of the artificial collagen to be used in the present invention is ideally set to a thickness of 1 to 20 mm. Setting the shape to any shape within the range is suited from the viewpoints of, for example, growth property of cartilage cells, strength, and handling convenience. Further, such a structure that the artificial collagen is conjugated (e.g., attached or laminated) with a lactic acid-caprolactone copolymer sponge may be adopted. In addition, an artificial collagen sponge to be used in the present invention may be used for repair of an osteochondral defect site in combination with hydroxyapatite, ceramics, or the like.

### Substrate for Cartilage Cultivation Containing Artificial Collagen

A substrate for cartilage cultivation of the present invention at least contains the above-mentioned artificial collagen. Further, the artificial collagen is preferably used in a form of a solution, in particular, an aqueous solution. The aqueous solution has a concentration of preferably 0.001% to 6.0% (W/V), more preferably 0.01% to 5.0% (W/V), further more preferably 0.05% to 3.0% (W/V), or most preferably 0.10% to 2.0% (W/V). Further, in the case of preparing a substrate having high consistency (containing artificial collagen in as high a concentration as 5% (W/V) or more), it is preferred to add an artificial collagen powder to a solution containing cartilage cells. It should be noted that an artificial collagen aqueous solution means a solution obtained by dissolving or partially dissolving artificial collagen in water or physiological saline.

In addition, the substrate for cartilage cultivation of the present invention may contain an additional active ingredient, a support or a carrier, or an additive, for example. Examples of the active ingredient include a bactericide or an antiseptic, an anti-inflammatory agent, an antiphlogistic analgesic agent, an antipruritic agent, an antiulcer agent, an antiallergic agent, an antivirus agent, an antifungal agent, antibiotics, an emollient agent, decubitus skin treatment agent, vitamin preparations, and herb medicine. Further, examples of the active ingredient include: sodium hyaluronate; growth factors such as basic fibroblast growth factor (bFGF), platelet-derived growth factor (PDGF), insulin, insulin-like growth factor (IGF), hepatocyte growth factor (HGF), glia-derived neurotrophic factor (GDNF), neurotrophic factor (NF), transforming growth factor (TGF), and vascular endothelial growth factor (VEGF); other cytokines such as bone morphogenetic protein (BMP) and transcription factors; hormones; inorganic salts such as Mg, Ca, and CO₃; organic substances such as citric acid and phospholipid; and medicaments such as anticancer agents. For the support or the carrier, various physiologically acceptable supports or carriers may be used depending on dosage forms (such as a solid formulation, a semisolid formulation, and a liquid formulation) of a biological material. Examples of the carrier for the solid formulation include a binder, an excipient, and a disintegrant.

In addition, examples of the carrier for the liquid formulation include water, alcohol (such as ethanol), ethylene glycol, propylene glycol, polyethylene glycol-polypropylene-glycol copolymer, and fat and oil (such as corn oil and olive oil).

### Applications of Substrate for Cartilage Cultivation

The substrate for cartilage cultivation of the present invention may be used for applications such as a scaffold material for cartilage cells, a material for differentiation and growth of cartilage cells, a material for cartilage regeneration, a material for promoting cartilage regeneration and protective material for joint function. For example, the substrate for cartilage cultivation of the present invention allows the construction of a cartilage tissue ex vivo and in vivo as a suitable scaffold for cartilage (cell) cultivation. That is, seeded cartilage cells use the substrate for cartilage cultivation as a scaffold, and tissue regeneration proceeds. Further, for example, if the substrate for cartilage cultivation of the present invention is grafted to a defective site of the cartilage, the regeneration (differentiation and growth) of a cartilage tissue is promoted at a grafted portion. In addition, for example, if the substrate for cartilage cultivation of the present invention is intraarticularly injected in and around a site of a cartilage transplantation of a patient after the cartilage transplantation, the regeneration of transplanted cartilage is promoted at an injection portion (proteoglycan production of transplanted cartilage cells can be enhanced by the injection). Also, for example, if the substrate for cartilage cultivation of the present invention is intraarticularly injected with in and around a cartilage defect site or a cartilage disorder site of a patient suffering with joint damage, the joint function of cartilage defect site or cartilage disorder site can be protected by the injection. A joint damage is exemplified by joint damage due to osteoarthritis, joint damage due to injury, joint damage due to sports, joint damage due to rheumatoid arthritis and joint damage due to connective tissue disorder such as systemic lupus erythematosus, but the exemplified joint damage is not particularly limited. A cartilage defect site means that a surface to deep portion of a construct gets severe damage, wherein the construct comprises hyaline cartilage and fibrocartilage and faces joint cavity. A cartilage disorder site means that a surface of a construct comprising hyaline cartilage and fibrocartilage and facing joint cavity gets minimal damage.

Further, the form and shape of a scaffold material and an implant for each of which the substrate for cartilage cultivation of the present invention is applied are not particularly limited, and any form and shape such as a sponge, a mesh, a nonwoven fabric formed product, and disk, film, rod, particle, and paste forms and shapes may be used. Such form and shape may be appropriately selected depending on the purpose of use of the scaffold material and the implant.

### Method for Cartilage Regeneration Treatment, Method for Cartilage Regeneration-Promotion Treatment and Method for Protecting Joint Function Using Substrate for Cartilage Cultivation

For a method of administrating for the substrate for cartilage cultivation of the present invention, the following methods are exemplified.

1 When the substrate for cartilage cultivation of the present invention is administered by the intraarticular injection rather than direct grafting to a cartilage defect site and an osteochondral defect site, the following administration method is suitably employed. The substrate for cartilage cultivation containing artificial collagen is intraarticularly administered in an amount of 0.1 mg to 300 mg with respect to 1 cm³ of a solution. In particular, the artificial collagen is administered in and around the joint with a cartilage defect. In addition, 1×10⁵ to 1×10⁷ cartilage cells may be contained with respect to 1 cm³ of the solution.

2 When grafting is performed by incising the joint and when grafting is performed with a cartridge type syringe under arthroscopy (direct grafting to a cartilage defect site and an osteochondral defect site is performed), the following administration method is suitably employed. The artificial collagen is administered to a cartilage defect site and/or an osteochondral defect site in an amount of 0.1 mg to 1000 mg with respect to 1 cm³ of a cartilage defect and/or an osteochondral defect. In addition, 1×10⁶ to 3×10⁸ cartilage cells may be contained with respect to 1 cm³ of the cartilage defect or the osteochondral defect. Also, for a patient after the cartilage transplantation, the artificial collagen is administered in and around a cartilage transplantation of the patient. In addition, for a patient suffering with joint damage, the artificial collagen is administered in and around a cartilage defect site or a cartilage disorder site of the patient. It should be noted that the consistency may be changed by changing the concentration of artificial collagen. For example, if changing the consistency of artificial collagen, a substrate for cartilage cultivation with high, moderate or low viscosity can be prepared. And, a substrate for cartilage cultivation having varying consistency can be administered to a cartilage defect site or an osteochondral defect site of a patient. Table 1 below shows examples that a substrate for cartilage cultivation having varying consistency being administered to a cartilage defect site or an osteochondral defect site of the patient. With regard to the osteochondral defect, grafting may be performed on a base part throughout which hydroxyapatite or ceramic granules or blocks have been spread.

### Cartilage Cells

Cartilage cells for use in the present invention may be obtained from cell sources involving allogeneic or autologous cells isolated from the joint cartilage, periosteum, and perichondrium, and mesenchymal (stromal) stem cells derived from the bone marrow. Because the allogeneic cells have a potential relating to an immune response and an infectious complication, the cartilage cells are preferably isolated from the autologous cells, in particular, autologous joint cartilage. Techniques for harvesting cells have been already known and have included enzyme digestion or outgrowth cultivation. The harvested cells are then multiplied in cell cultivation prior to implantation in the body. In general, in order to provide optimum regeneration of a cartilage tissue, at least 10⁶, or preferably at least 10⁷ cells respect to 1 cm³ of a solution should be impregnated into a substrate for cartilage cultivation.

### Proteoglycan

A proteoglycan is a protein having a glycosaminoglycan (GAG) as a sugar side chain, and has been called a mucopolysaccharide in ancient times. The proteoglycan is present in a large amount in connective tissues such as bone, cartilage, and skin, and is present in a cell or in a cell membrane.

### Aggrecan

An aggrecan is a large chondroitin sulfate proteoglycan which forms a majority of proteoglycans contained in a cartilage tissue, is present in a large amount in the cartilage, and occasionally accounts for 50% (W/W) of the tissue dry weight.

### Type 2 Collagen

Type 2 collagen is a major component which accounts for about 50% (W/W) of the cartilage dry weight. That is, high expression of Type 2 collagen mRNA means that the production of extracellular matrix is promoted in cartilage cells.

### SOX9

SOX9 is a DNA binding transcription factor having a high-mobility-group (HMG) domain, and is said to be essential for aggregation of mesenchymal cells and transformation into cartilage cells.

### RGD

An arginine-glycine-aspartic acid (RGD: SEQ ID NO: 1) sequence is found in some important extracellular matrix proteins, and serves as an adhesion ligand to an integrin family member of a cell surface receptor. A typical RGD sequence is Gly-Arg-Gly-Asp-Ser-Pro (GRGDSP; SEQ ID NO: 2). A cyclic RGD may also be used as a cell adhesion motif. Another typical sequence is Arg-Gly-Asp-(D-Phe)-Val (RGDFV: SEQ ID NO: 3). An RGD modified surface leads the formation of a cell single layer in situ on a membrane. That is, it is conceivable that the substrate for cartilage cultivation of the present invention contains any one of the RGD peptides as described above, and thus can enhance an adhesion ability to cartilage cells and can enhance a growth ability of cartilage cells.

### Joint Function Improving Agent

The intraarticular injection of a hyaluronic acid formulation having a cartilage protection action is currently used to provide an effective therapeutic effect on diseases such as osteoarthritis, osteoarthritis after sport disorder, and rheumatoid arthritis. For example, in Japan, Souvenir® (manufactured and sold by Chugai Pharmaceutical Co., Ltd.) and Artz® (manufactured and sold by Seikagaku Corporation) are sold as a sodium hyaluronate intraarticular injection solution serving as a joint function improving agent for treatment of osteoarthritis and rheumatoid arthritis. Further, examples in foreign countries are as shown in Table 2 below. The usage and dose for the above-mentioned joint function improving agent are as described below. Knee osteoarthritis: in general, for an adult human, 2.5 ml (25 mg of sodium hyaluronate) is administered into the knee joint cavity once a week for 5 consecutive weeks. After that, when the maintenance of symptoms is intended, administration is performed at an interval of 2 to 4 weeks. Shoulder periarthritis: in general, for an adult human, 2.5 ml (25 mg of sodium hyaluronate) is administered into the shoulder joint (shoulder joint cavity, subacromial bursa, or sheath of long head of biceps tendon) once a week for 5 consecutive weeks. Knee joint pain in chronic rheumatoid arthritis: in general, for an adult human, 2.5 ml (25 mg of sodium hyaluronate) is administered into the knee joint cavity once a week for 5 consecutive weeks.

TABLE 2

| | Supartz | Synvisc | | | | |
|---|---|---|---|---|---|---|
| | (Artz) | (hylan G-F 20) | Hyalgan | Suflexxa | Orthovisc | Durolane |
| Manufacturer | Seikagaku | Genzyme | Fidia | Savient | Anika | Q-Med |
| Seller | Smith & Nephew | Genzyme | Sanofi- | Ferring Savient | DePuy Mitek | Q-Med |
| (United States) | | | Synthelabo | | | (sold only in |
| | | | | | | Europe) |
| Indication* | Knee | Knee | Knee | Knee | Knee | Knee |
| | osteoarthritis | osteoarthritis | osteoarthritis | osteoarthritis | osteoarthritis | osteoarthritis |
| Administration | Once a week for | Once a week for | Once a week for | Once a week for | Once a week for | Once |
| method | 5 consecutive | 3 consecutive | 5 consecutive | 3 consecutive | 3 to 4 consecutive | |
| | weeks | weeks | weeks | weeks | weeks | |
| Date of | January, 2001 | August, 1997 | May, 1997 | December, 2004 | February, 2004 | Unapproved |
| FDA approval | | | | | | (sold only in |
| (Medical device) | | | | | | Europe) |
| Composition | HA 1% 2.5 mL | HA (Hylan G-F20) | HA 1% 2 mL | HA 1% | HA 1% 2 mL | HA (NASHA |
| | | 0.8% 2 mL | | | | Gel) 2% 3 mL |
| Origin of HA | Cockscomb | Cockscomb | Cockscomb | Non-animal | Cockscomb | Non-animal |
| | | | | | | (NASHA) |
| Molecular | 600,000 to | 6,000,000 | 500,000 to | 2,400,000 to | 1,000,000 to | Cross-linked |
| weight of HA | 1,200,000 | or more | 730,000 | 3,600,000 | 2,900,000 | HA |
| | | Cross-linked | | Non-cross- | | |
| | | HA chemically | | linked | | |
| | | modified with | | | | |
| | | formaldehyde | | | | |
| | | etc. | | | | |

*A case where pain is not sufficiently suppressed by rehabilitation or a mild analgesic agent such as acetaminophen

The artificial collagen aqueous solution of the present invention has a cartilage repair effect. In order to repair the cartilage of joints in the whole body such as a knuckle joint having a small joint volume, and a knee joint and a hip joint each having a large joint volume, it is preferred to perform administration while changing the concentration within the range of 0.1 mg/ml to 300 mg/ml. Further, mixing an artificial collagen aqueous solution with hyaluronic acid or the like may enhance a cartilage protection effect and a cartilage repair effect. The cartilage as described here includes fibrocartilages such as hyaline cartilage and meniscus (knee joint), and glenoid labrum (hip joint). That is, it is conceivable that a joint function improving agent prepared by adding the artificial collagen of the present invention to hyaluronic acid or the above-mentioned joint function improving agent is intraarticularly administered to provide a cartilage protection effect and a cartilage repair promoting effect, as recognized from the results of the following examples. Further, the joint function improving agent of the present invention may be manufactured by adding the artificial collagen to 2.5 ml of the above-mentioned joint function improving agent in an amount of 0.1 mg to 300 mg with respect to 1 cm³ of a solution.

When the substrate for cartilage cultivation or the joint function improving agent of the present invention is used for medical applications, disinfection or sterilization is preferably performed before use. For disinfection and sterilization methods, there are exemplified various disinfection and sterilization methods such as moist heat and steam sterilization, gamma sterilization, ethylene oxide gas sterilization, chemical disinfection, and ultraviolet radiation disinfection. Of those methods, gamma sterilization and ethylene oxide gas sterilization are preferred because of high sterilization efficiency and a small influence on a material.

The substrate for cartilage cultivation or the joint function improving agent of the present invention may be applied to tissues (for example, an epidermal tissue and a dermal tissue) of various subjects (patients). The subjects (patients) are not limited to a human, and may be non-human animals (for example, non-human animals such as a monkey, a sheep, an ox or a cow, a horse, a dog, a cat, a rabbit, a rat, and a mouse).

Hereinafter, the present invention is clarified more specifically by way of examples of the present invention. It goes without saying that the present invention is not in any way limited to the description of those examples. Further, it should be appreciated that, in the present invention, various changes, modifications, and improvements may be made based on the knowledge of those skilled in the art without departing the gist of the present invention in addition to the following examples and the embodiments of the present invention as described above.

### EXAMPLE 1

### Confirmation of Effect of Scaffold for Cartilage Cultivation Containing Artificial Collagen

Confirmation was made on an effect of a scaffold for cartilage (cell) cultivation containing the artificial collagen of the present invention. The details are as follows.

### Artificial Collagen

The artificial collagen used in the present invention is artificial collagen sold by PHG Corporation (INCI name: Poly(Tripeptide-6), CAS. No: 60961-94-6: www.phg.co.jp/research/collagen.html).

### Preparation of Cartilage Cells

Shoulder joints and knee joints were extirpated from 5 New Zealand white rabbits (male, 8- to 9 week-old), and only a cartilage layer was excised and sampled with a surgical knife. After washing with a sterilized PBS solution, pronase was added to 25 ml of Dulbecco's Modified Eagle's medium+gentacin (25 μg/ml) at a concentration of 0.4% (W/V), and incubation was performed at 37° C. for 2 hours. The solution was supplemented with collagenase at a concentration of 0.025% (W/V), and incubated at 37° C. overnight. The resulting solution was washed to collect cartilage cells. After that, the cartilage cells were seeded into a dish with a diameter of 90 mm at 1 to 2×10⁶ cells/dish, and a medium exchange was performed once every 3 days. At the time point where the cells became confluent, subcultivation was performed only once. The composition of a cultivation medium is as described below.

### Composition of Cultivation Medium

Dulbecco's Modified Eagle's medium nutrient mixture F-12 HAM (SIGMA)+10% FETAL BOVINE SERUM (Hyclone)+20 μg/ml ascorbic acid (SIGMA). It should be noted that the following experiments were performed by using cartilage cells which had been subcultivated once.

Cultivation of Cartilage Cells with Collagen

One piece of artificial collagen sponge (10 mm×5 mm×7 mm) was attached to one well in a 24-well cultivation tray, and subjected to ethylene oxide gas sterilization. The cells were injected into the artificial collagen sponge so that 5×10⁵ cells would be contained in 40 μl. The cells were subsequently left to stand in an incubator for 1 hour, and then supplemented with 2 ml of a cultivation medium. It should be noted that the composition of the cultivation medium in cultivation is as described below.

### Composition of Cultivation Medium

Dulbecco's Modified Eagle's medium nutrient mixture F-12 HAM (SIGMA)+20% FETAL BOVINE SERUM (Hyclone)+20 μg/ml ascorbic acid (SIGMA). It should be noted that a medium exchange was performed once every 3 days.

Meanwhile, as a control, 2 ml of bovine type 2 collagen (2% (W/V)) manufactured by Nitta Gelatin Inc. were charged into each of 24 wells, and subjected to lyophilization and ethylene oxide gas sterilization. The cells were seeded so that 5×10⁵ cells would be contained in 1 ml. It should be noted that the composition of the cultivation medium in cultivation is as described below.

### Composition of Cultivation Medium

Dulbecco's Modified Eagle's medium nutrient mixture F-12 HAM (SIGMA)+20% FETAL BOVINE SERUM (Hyclone)+20 μg/ml ascorbic acid (SIGMA). It should be noted that a medium exchange was performed once every 3 days.

### Preparation of Toluidine Blue Stained Specimen

Each of samples after 1 week, 2 weeks, and 3 weeks of cultivation was collected and fixed with formalin to prepare a tissue specimen, and the tissue specimen was stained with toluidine blue.

### Measurement of Proteoglycan Amount, DNA Amount, and Gene Expression Amount of Aggrecan, Type 2 Collagen, and Sox9

Each of samples after 1 week, 2 weeks, and 3 weeks of cultivation was collected and cryopreserved at −70° C., the proteoglycan amount was measured by a dimethylmethlene blue (DMMB) method (whose details are described in the following paragraph), and the DNA amount was measured by a DNA assay (whose details are described in the following paragraph). It should be noted that the collected sample was supplemented with 400 μL of a 60 μg/mL papain digestion solution and incubated at 58° C. overnight.

### Composition of Papain Digestion Solution

Papain buffer 25 mL+25 mg/ml papain stock (60 μl)+L-cysteine hydrochloride monohydrate (Wako) 21.95 mg. Papain buffer=0.1 M sodium acetate, 0.05 M EDTA, pH 5.53 in D.W. 25 mg/ml papain stock=papain (Wako) in papain buffer. In addition, the gene expression amount of an aggrecan, type 2 collagen, and sox9 was measured (the details are described in the following paragraph).

### Method for Measurement of Proteoglycan Amount

75 μL each of the above-mentioned sample digested with papain and a proteoglycan standard (Bovine Nasal Septum) adjusted in the range of 2.12 μg to 16.0 μg were used, and 25 μL of 2.88 M GuHCL were added thereto. After that, 200 μL of a 16 μg/mL dimethylmethlene blue solution were added thereto, and the mixture was shaken under a light-shielding condition for 30 seconds. The absorbances were measured at 530 nm and 590 nm, and the proteoglycan amount in the sample was quantitatively determined from a calibration curve prepared by using the proteoglycan standard.

### Method for Measurement of DNA Amount

100 μL each of the above-mentioned sample digested with papain and a DNA standard (Calf thumus DNA) adjusted in the range of 0.08 μg to 40 μg were used, and 100 μL of a 1 μg/mL Hoechst 33258 dye solution were added thereto. The mixture was shaken under a light-shielding condition for 30 seconds. Then, fluorescence (excitation 360 and emission 460) was measured. The DNA amount in the sample was quantitatively determined from a calibration curve prepared by using the DNA standard.

### Method for Measurement of Gene Expression Amount of Aggrecan, Type 2 Collagen, and Sox9

RNA was extracted from each of samples on Week 1, Week 2, and Week 3 of the artificial collagen sponge and the bovine type 2 collagen sponge to prepare cDNA, and real-time PCR was performed for aggrecan, type 2 collagen, and sox9. The model used was ABI PRISM 7000 (Applied Biosystems), and the reagents used were Real-time PCR Master Mix (TOYOBO) and Pre-Developed TaqMan & reg; Assay Reagents Eukaryotic 18S rRNA (Applied Biosystems), TaqMan & reg; Probe kit (Applied Biosystems).

The following are the used primer sequence and reaction condition.

### (Used Primer)

<Aggrecan>

Rabbit AGGR-F:
(SEQ ID NO: 4)
5′-GATCTACCGCTGTGAGGTGATG-3′
Rabbit AGGR-R:
(SEQ ID NO: 5)
5′-CCTTTCACCACGACCTCCAA-3′
TaqMan & reg; probe:
(SEQ ID NO: 6)
5′-ACGGCCTTGAGGACAGCGAGGCTAC-3′

<type 2 collagen>

Rabbit COL2-F:
(SEQ ID NO: 7)
5′-CCCCCGCTCTCCAAGAGA-3′
Rabbit COL2-R:
(SEQ ID NO: 8)
5′-GCCAGGAAGACAATAAATAAATAGAACA-3′
TaqMan probe:
(SEQ ID NO: 9)
5′-TGAACTGGGCAGACTGCAAAACAAAAGCT-3′

<Sox9>

Rabbit SOX9-F:
(SEQ ID NO: 10)
5′-AGTACCCGCACCTGCACAA-3′
Rabbit SOX9-R:
(SEQ ID NO: 11)
5′-CGCTTCTCGCTCTCGTTCAG-3′
TaqMan probe:
(SEQ ID NO: 12)
5′-AGCTCAGCAAGACCCTCGGGAAGC-3′

<Reaction Condition>

At 50° C. for 2 minutes, at 95° C. for 10 minutes, (at 95° C. for 15 seconds, at 60° C. for 1 minute)×40 cycles.

### Observation Results of Toluidine Blue Stained Specimen

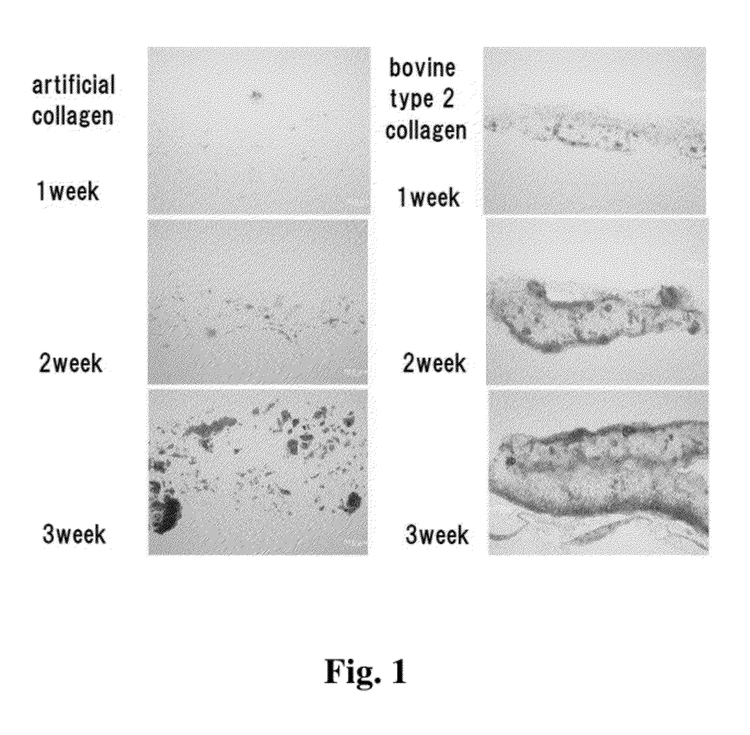

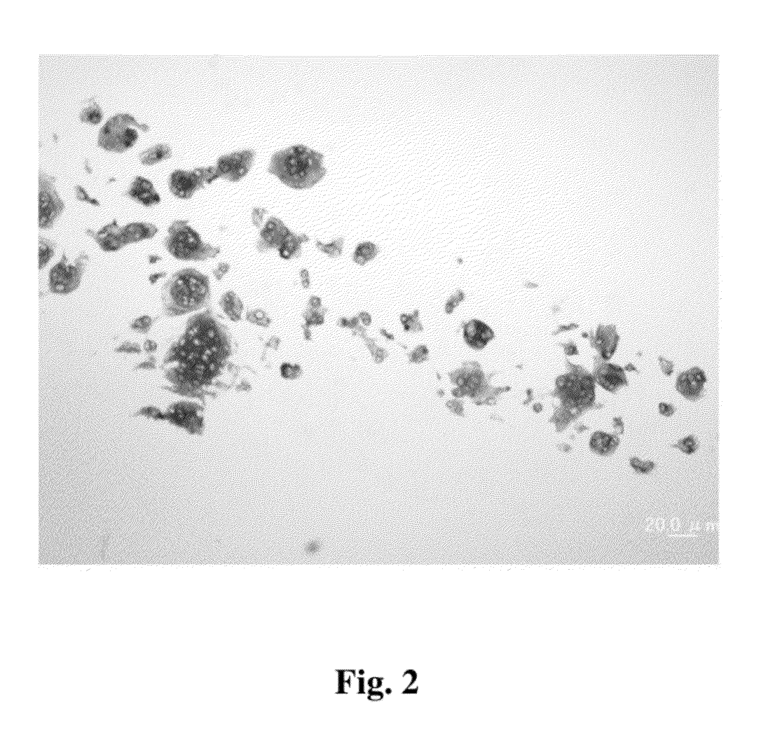

FIG. 1 and FIG. 2 show the observation results of toluidine blue stained specimens. As can be seen from FIG. 1, the artificial collagen sponge showed rapid growth of cartilage cells over a period from Week 2 to Week 3. Further, FIG. 2 shows an enlarged photograph of the artificial collagen sponge on Week 3, and cartilage cells could be confirmed from the photograph.

### Measurement Results of Proteoglycan Amount

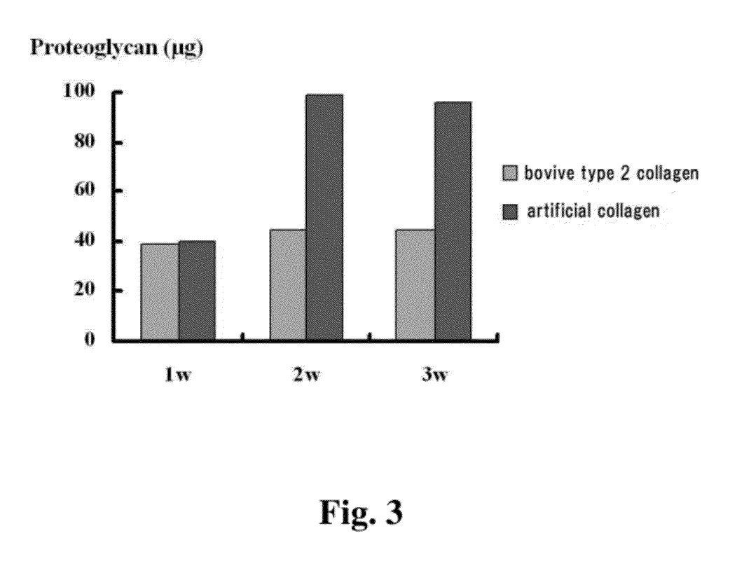

FIG. 3 illustrates the measurement results of the proteoglycan amount.

As can be seen from FIG. 3, when cartilage cultivation was performed in the artificial collagen sponge, the proteoglycan amount was 39.7 μg on Week 1, and was increased to 90 μg or more on Week 2 and Week 3. Meanwhile, when cultivation was performed in the bovine type 2 collagen sponge, only a slight increase in proteoglycan amount was observed on Week 2 and Week 3.

### Measurement Results of DNA Amount

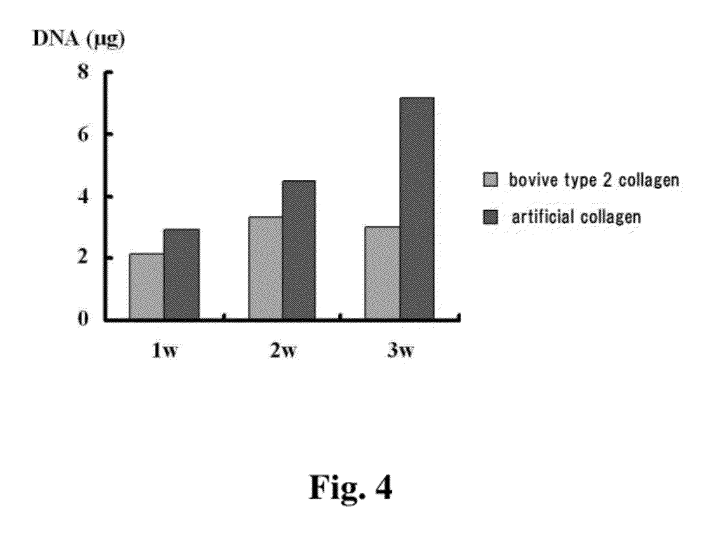

FIG. 4 illustrates the measurement results of the DNA amount. As can be seen from FIG. 4, when cartilage cultivation was performed in the artificial collagen sponge, the DNA amount was 2.9 μg on Week 1, and was increased to 4.5 μg on Week 2 and 7.2 μg on Week 3. Meanwhile, when cultivation was performed in the bovine type 2 collagen sponge, the DNA amount was not increased on Week 2 and Week 3.

### Measurement Results of Proteoglycan/DNA Ratio

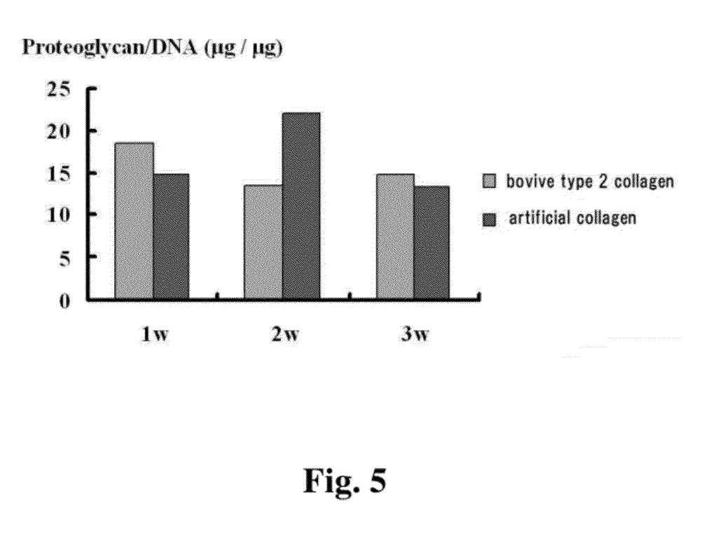

FIG. 5 illustrates the ratio of the proteoglycan amount to the DNA amount. As can be seen from FIG. 5, when cultivation was performed in the artificial collagen sponge, the proteoglycan/DNA ratio was 13.3 μg/μg. That is, when cultivation was performed in the artificial collagen sponge, the proteoglycan/DNA ratio was almost comparable to the proteoglycan/DNA ratio of 14.7 μg/μg measured when cultivation was performed in bovine type 2 collagen sponge. Bovine type 2 collagen is currently used as a scaffold for cartilage grafting. Because the proteoglycan/DNA ratio of artificial collagen is almost comparable to the proteoglycan/DNA ratio of the bovine type 2 collagen, the artificial collagen sponge may also be used for cartilage grafting.

### Measurement Results of Gene Expression Amount of Aggrecan, Type 2 Collagen, and Sox9

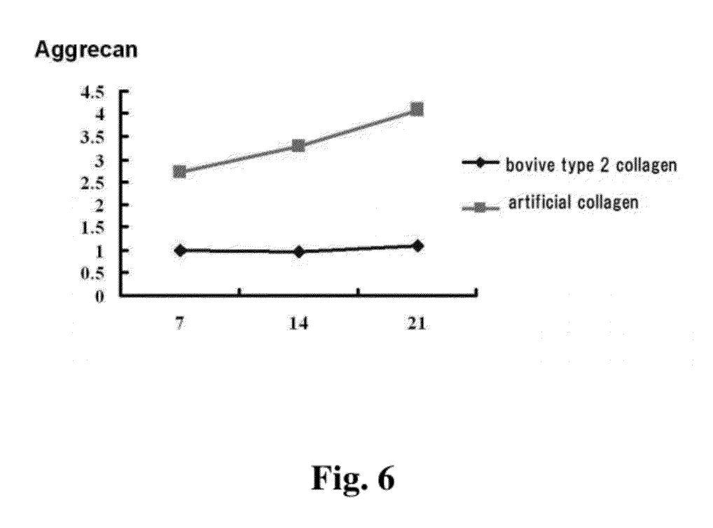

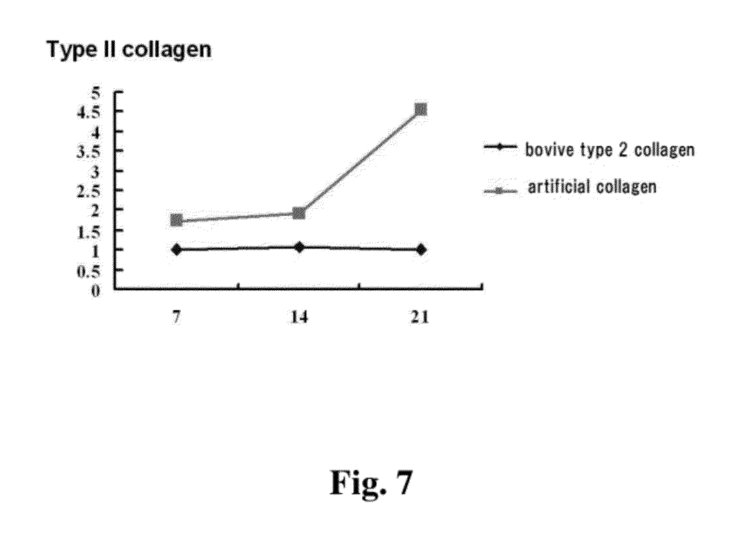

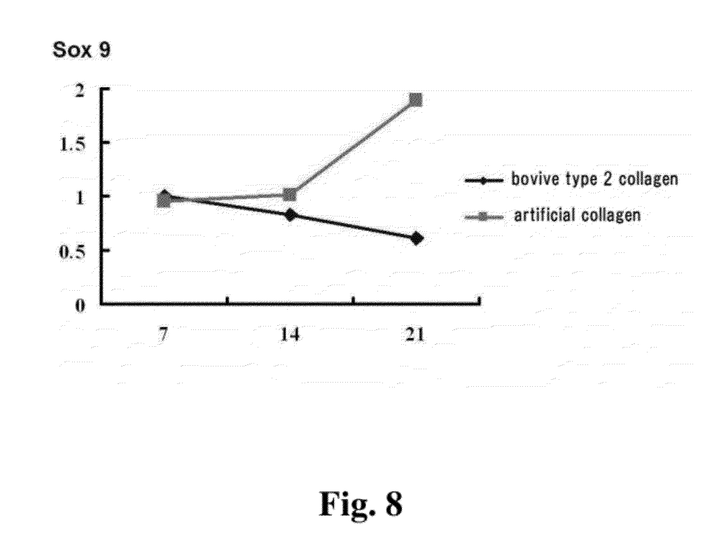

FIGS. 6 to 8 illustrate the gene expression amount of an aggrecan, the gene expression amount of type 2 collagen, and the gene expression amount of sox9, respectively. The numerical values in the figures are represented by a relative evaluation in which a value on Week 1 in the bovine type 2 collagen sponge, which was measured by real-time PCR, was defined as 1. The gene expression of an aggrecan and type 2 collagen each forming extracellular matrix in the cartilage was not changed on Week 2 and Week 3 in the bovine type 2 collagen sponge, while was increased on Week 2 and Week 3 in the artificial collagen sponge. Further, the gene expression of sox9 associated with differentiation of the cartilage was decreased on Week 2 and Week 3 in the bovine type 2 collagen sponge, while was increased in the artificial collagen sponge. Thus, the measurement results of the gene amount relating to the gene expression also revealed that the artificial collagen sponge was suitable for cartilage cultivation.

### General Statement on Results of Example 1

The results of Example 1 above suggest the following. The artificial collagen sponge of the present invention has no antigenicity unlike collagen of biological origin, and thus can be used as a scaffold for cartilage cultivation and grafting which has no risk of infection with a virus and a prion. The artificial collagen of the present invention is not of biological origin, and hence can be used as a scaffold for evaluation on a cartilage growth factor and a proteoglycan growth factor. It is conceivable that the artificial collagen of the present invention contains an RGD peptide, and thus can enhance an adhesion ability to cartilage cells and can enhance a growth ability of cartilage cells. Further, the artificial collagen of the present invention contains a cell growth promoter, and thus can be used for evaluation on effectiveness of the promoter. It is conceivable that cartilage regeneration can be promoted by mixing the artificial collagen of the present invention with hyaluronic acid having a cartilage protection action, and adding the mixture to a grafting site.

### EXAMPLE 2

### Confirmation of Effect of Artificial Collagen Aqueous Solution

Confirmation was made on an effect of an aqueous solution containing the artificial collagen of the present invention on cartilage cells. The details are as follows.

### Artificial Collagen

The artificial collagen used in the present invention is artificial collagen sold by PHG Corporation (INCI name: Poly(Tripeptide-6), CAS. No: 60961-94-6: www.phg.co.jp/research/collagen.html).

### Preparation of Cartilage Cells

Shoulder joints and knee joints were extirpated from 5 New Zealand white rabbits (male, 8- to 9 week-old), and only a cartilage layer was excised and sampled with a surgical knife. After washing with a sterilized PBS solution, pronase was added to 25 ml of Dulbecco's Modified Eagle's medium+gentacin (25 μg/ml) at a concentration of 0.4% (W/V), and incubation was performed at 37° C. for 2 hours. The solution was supplemented with collagenase at a concentration of 0.025% (W/V), and incubated at 37° C. overnight. The resulting solution was washed to collect cartilage cells. After that, the cartilage cells were seeded into a dish with a diameter of 90 mm at 1 to 2×10⁶ cells/dish, and a medium exchange was performed once every 3 days. At the time point where the cells became confluent, subcultivation was performed only once. The following experiments were performed by using cells which had been subcultivated once. Further, the composition of a cultivation medium is as described below.

### Composition of Cultivation Medium

Dulbecco's Modified Eagle's medium nutrient mixture F-12 HAM (SIGMA)+20% FETAL BOVINE SERUM (Hyclone)+20 μg/ml ascorbic acid (SIGMA) were used, and a medium exchange was performed once every 3 days.

### Analysis of Proteoglycan Production Ability of Cartilage Cells

Measurement was made on a proteoglycan production ability of cartilage cells by addition of the aqueous solution containing the artificial collagen of the present invention. The details are as follows.

### 1 Seeding of Cartilage Cells

Cartilage cells in a semi-confluent state were detached from a dish by a tripsin-EDTA solution treatment, supplemented with a cultivation medium, and washed by centrifugation. After that, the cells were seeded into a 6-well plate at 1.5×10⁶ cells/well and cultivated under conditions of 37° C. and 5% CO₂.

### 2 Addition of Artificial Collagen Aqueous Solution

At 24 hours after the start of the cultivation, a 1% (W/V) artificial collagen aqueous solution was diluted with a cultivation medium to prepare 0.10, 0.20, 0.30, 0.40, and 0.50% (W/V) artificial collagen aqueous solutions. The artificial collagen aqueous solutions were allowed to act on the cells for an additional 7 days, and further supplemented with 10 μCi/ml Na₂³⁵SO₄ to perform cultivation for the last 24 hours. A medium exchange was performed once every 3 days. It should be noted that a 0% (W/V) artificial collagen aqueous solution was used as a control.

### 3 Collection and Extraction

After the completion of the above-mentioned cultivation, the cells were washed with a fresh cultivation medium, and a lysis buffer (4 M GuHCl, 0.05 M NaAC, pH 6.0) supplemented with protease inhibitors (0.1 M 6-aminohexanoic acid, 0.005 M benzamidine hydrochloride, 0.01 M Naₑ EDTA, 0.01 M N-ethylmaleimide, and 0.001 M phenylmethyl sulfonyl fluoride) was added into each well. The cells were extracted at 4° C. for 4 hours, and centrifuged at 15000 rpm at 4° C. for 20 minutes.

### 4 Separation and Radioactivity Measurement

The cells were eluted with an elution buffer (4 M GuHCl, 0.05 M Na acetate, 0.1 M Na sulfate, and 0.5% Triton X-10 (pH 7.5)) by using a PD-10 pre-packed column (GE Healthcare Bio-Sciences Ltd.) to fractionate samples. To each of the fractionated samples, ethanol and a scintillator were added and mixed, and the radioactivity content (cpm) was measured with a liquid scintillation counter. Further, the DNA amount of each of the samples used in this case was measured with a Hoechst 33258 dye to determine a DNA value expressed as cpm/mg.

Measurement of mRNA Amount of Type 2 Collagen and Aggrecan in Cartilage Cells

Measurement was performed on the amount of the type 2 collagen mRNA and the aggrecan mRNA in cartilage cells by addition of the aqueous solution containing the artificial collagen of the present invention. The details are as follows.

### 1 Seeding of Cartilage Cells

Cartilage cells in a semi-confluent state were detached from a dish by a tripsin-EDTA solution treatment, supplemented with a cultivation medium, and then washed by centrifugation. After that, the cells were seeded into a 12-well plate at 3×10⁵ cells/well, and cultivation was started under conditions of 37° C. and 5% CO₂.

### 2 Addition of Artificial Collagen Aqueous Solution

At 24 hours after the start of the cultivation, a 1% (W/V) artificial collagen aqueous solution was diluted with a cultivation medium to prepare 0.10, 0.20, 0.30, 0.40, and 0.50% (W/V) artificial collagen aqueous solutions. Then, cultivation was performed for 4, 7, 14, and 21 days. It should be noted that a 0% (W/V) artificial collagen aqueous solution was used as a control.

### 3 Collection and RNA Extraction of Cartilage Cells

After the completion of the above-mentioned cultivation, the cartilage cells were collected, and RNA extraction was performed by using RNeasy (registered trademark) Mini Kit (QIAGEN). A reverse transcription reaction was performed by using QuantiTect (registered trademark) Reverse Transcription Kit (QIAGEN). To be more specific, Real-time PCR was performed by the following method.

<Real-time PCR>

The model used for Real-time PCR was ABI PRISM 7000 (Applied Biosystems), and the reagents used were Real-time PCR Master Mix (TOYOBO) and Pre-Developed TaqMan & reg; Assay Reagents Eukaryotic 18S rRNA (Applied Biosystems), TaqMan & reg; Probe kit (Applied Biosystems).

In addition, the following are the used primer sequence and reaction condition.

<Aggrecan>

Rabbit AGGR-F:
(SEQ ID NO: 4)
5′-GATCTACCGCTGTGAGGTGATG-3′
Rabbit AGGR-R:
(SEQ ID NO: 5)
5′-CCTTTCACCACGACCTCCAA-3′
TaqMan & reg; probe:
(SEQ ID NO: 6)
5′-ACGGCCTTGAGGACAGCGAGGCTAC-3′

<Type 2 Collagen>

Rabbit COL2-F:
(SEQ ID NO: 7)
5′-CCCCCGCTCTCCAAGAGA-3′
Rabbit COL2-R:
(SEQ ID NO: 8)
5′-GCCAGGAAGACAATAAATAAATAGAACA-3′
TaqMan probe:
(SEQ ID NO: 9)
5′-TGAACTGGGCAGACTGCAAAACAAAAGCT-3′

<Reaction Condition>

At 50° C. for 2 minutes, at 95° C. for 10 minutes (at 95° C. for 15 seconds, at 60° C. for 1 minute)×40 cycles.

### Analysis Results of Proteoglycan Production Ability of Cartilage Cells

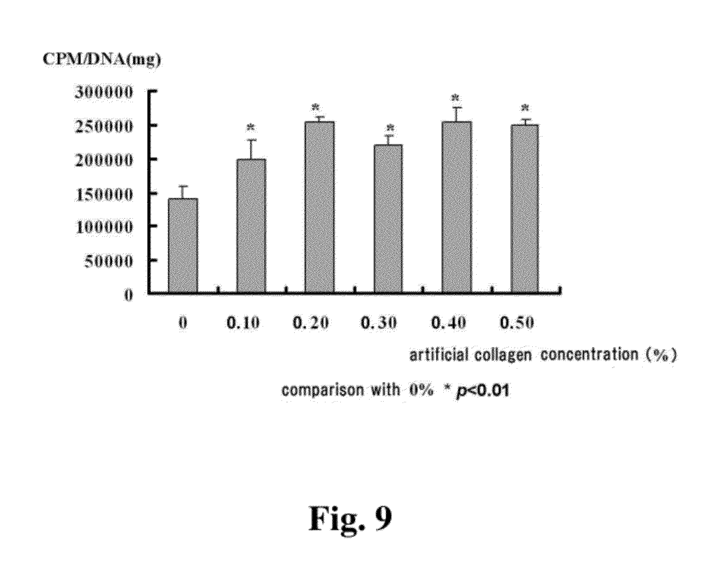

FIG. 9 illustrates the analysis results of the proteoglycan production ability of cartilage cells by addition of the artificial collagen aqueous solution. As illustrated in FIG. 9, the proteoglycan production was significantly enhanced by addition of 0.10% (W/V), 0.20% (W/V), 0.30% (W/V), 0.40% (W/V), and 0.50% (W/V) artificial collagen aqueous solutions.

Measurement Results of type 2 Collagen mRNA Amount and Aggrecan mRNA Amount in Cartilage Cells

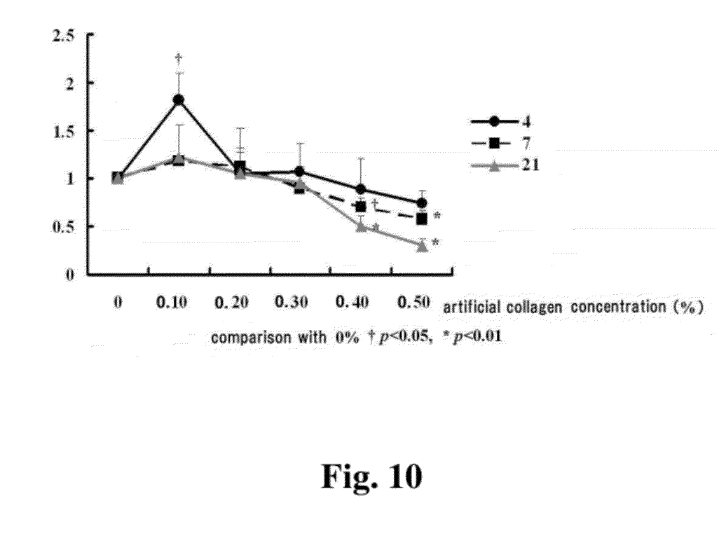

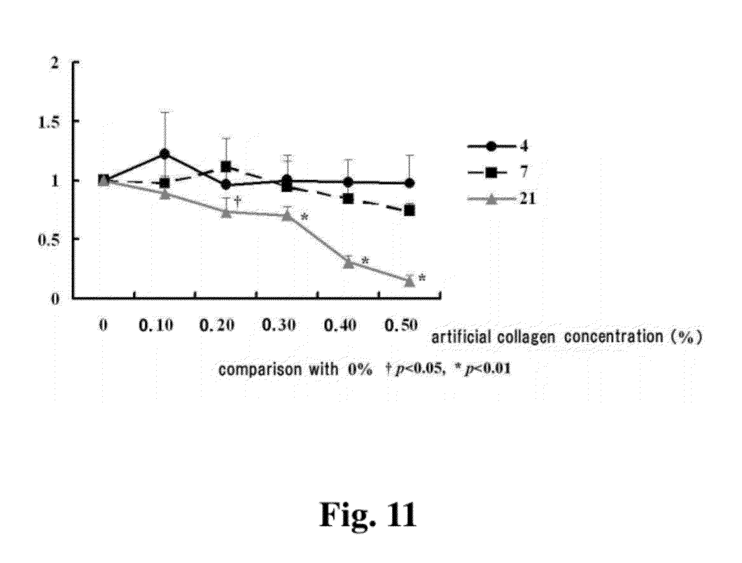

FIG. 10 and FIG. 11 illustrate the measurement results of the type 2 collagen mRNA amount and the aggrecan mRNA amount in cartilage cells by addition of the artificial collagen aqueous solution, respectively. As illustrated in FIG. 10, on Day 4, mRNA expression was significantly enhanced in a group in which a 0.10% (W/V) artificial collagen aqueous solution was added. Further, on Day 7 and Day 21, mRNA expression was lowered in groups to which 0.40% (W/V) and 0.50% (W/V) artificial collagen aqueous solutions were added. As illustrated in FIG. 11, on Day 4 and Day 7, there was no significant change in aggrecan mRNA amount in groups having artificial collagen aqueous solutions with the respective concentrations added. On Day 21, mRNA was significantly lowered in groups in which artificial collagen aqueous solutions having concentrations of 0.20% (W/V), 0.30% (W/V), 0.40% (W/V), and 0.50% (W/V) were added. It should be noted that a relative evaluation was performed by defining an RNA amount measured for a 0% artificial collagen aqueous solution on each time point as 1.

### General Statement of Results of Example 2

The results of Example 2 above suggest the following. The addition of the artificial collagen aqueous solution of the present invention to cartilage cells allows a proteoglycan production ability and a type 2 collagen synthesis ability of cartilage cells to be enhanced without impairing a DNA synthesis ability (growth ability) and an aggrecan mRNA synthesis ability of cartilage cells. The artificial collagen aqueous solution of the present invention has no antigenicity unlike collagen of biological origin, also has no risk of infection with a virus and a prion, and hence may be used for cartilage cell cultivation and grafting. In addition, the artificial collagen aqueous solution of the present invention may be used for cartilage repair through a direct intraarticular administration to a human. In addition, the artificial collagen aqueous solution of the present invention also has an effect of promoting the cartilage repair. It is conceivable that the artificial collagen aqueous solution of the present invention contains an RGD peptide, and thus can enhance an adhesion ability to cartilage cells, and further can enhance a proteoglycan production ability and a type 2 collagen synthesis ability in cartilage cells.

### EXAMPLE 3

### Confirmation of Cartilage Regeneration Effect by Intraarticular Administration of Substrate for Cartilage Cultivation of Present Invention)

Confirmation was made whether a cartilage defect site was regenerated (repaired) by the intraarticular administration of the substrate for cartilage cultivation containing the artificial collagen aqueous solution of the present invention. The details are as follows.

### Artificial Collagen

The artificial collagen used in the present invention is artificial collagen sold by PHG Corporation (INCI name: Poly(Tripeptide-6), CAS. No: 60961-94-6: www.phg.co.jp/research/collagen.html).

### Preparation of Cartilage Defect Model

A cartilage defect with a diameter of 5 mm was made on the patellofemoral joint of each of 18 knees of 9 New Zealand white domestic rabbits (22 week-old, 3.4 to 3.8 kg) after anesthesia, and further, a wound was sutured to prepare a cartilage defect model (Week 0).

### Intraarticular Administration of Artificial Collagen Aqueous Solution in Cartilage Defect Model

The 18 knees were divided into 3 groups, and on Week 1, Week 3, and Week 5, physiological saline was administered for a control group, and an intraarticular injection (0.1 ml/kg) was performed for an artificial collagen aqueous solution administration group. After that, knee joints were collected on Week 7.

The details are as described below and shown in Table 3.

Control group (N=6): physiological saline

0.1% (W/V) group (N=6): 0.1% (W/V) artificial collagen aqueous solution

0.5% (W/V) group (N=6): 0.5% (W/V) artificial collagen aqueous solution

TABLE 3

| | Week 0 | Week 1 | Week 3 | Week 5 | Week 7 |
|---|---|---|---|---|---|
| Control | Cartilage | Physio- | Physio- | Physio- | Knee joint |
| group | defect | logical | logical | logical | collection |
| | preparation | saline | saline | saline | |
| | | admin- | admin- | admin- | |
| | | istration | istration | istration | |
| 0.1% | Cartilage | Intra- | Intra- | Intra- | Knee joint |
| group | defect | articular | articular | articular | collection |
| | preparation | admin- | admin- | admin- | |
| | | istration | istration | istration | |
| 0.5% | Cartilage | Intra- | Intra- | Intra- | Knee joint |
| group | defect | articular | articular | articular | collection |
| | preparation | admin- | admin- | admin- | |
| | | istration | istration | istration | |

Evaluation with Modified ICRS Score

The ICRS score in Table 4 below is an evaluation method generally used when cartilage grafting to a cartilage defect is performed. The inventors of the present invention adopted, as an evaluation method in the case of administering a medicament without performing cartilage grafting, a modified ICRS score, which is an evaluation method involving using two items of “Degree of defect repair” and “Macroscopic appearance” and performing evaluation with a total score of 0 to 8. Table 5 below shows the results of Example 3.

TABLE 4
ICRS macroscopic evaluaton of cartilage repair

| Cartilage repair asessment ICRS | Points |
|---|---|
| Degree of defect repair | |
| In level with surrounding cartilage | 4 |
| 75% repair of defect depth | 3 |
| 50% repair of defect depth | 2 |
| 25% repair of defect depth | 1 |
| 0% repair of defect depth | 0 |
| Integration to border zone | |
| Complete integration with surrounding cartilage | 4 |
| Demarcating border <1 mm | 3 |
| ¾th of graft integrated, ¼th with a notable | 2 |
| border >1 mm width | |
| ½ of graft integrated with surrounding | 1 |
| cartilage, ½ with a notable border >1 mm | |
| From no contact to ¼th of graft integrated | 0 |
| with surrounding cartilage | |
| Macroscopic appearance | |
| Intact smooth surface | 4 |
| Fibrillated surface | 3 |
| Small, scattered fissures or cracs | 2 |
| Several, small or few but large fissures | 1 |
| Total degeneration of grafted area | 0 |
| Overall repair assessment | |
| Grade I: normal | 12 |
| Grade II: nearly normal | 11-8 |
| Grade III: abnormal | 7-4 |
| Grade IV: severely abnormal | 3-1 |

TABLE 5
Modified ICRS score

| Degree of defect repair | |
|---|---|
| In level with surrounding cartilage | 4 |
| 75% repair of defect depth | 3 |
| 50% repair of defect depth | 2 |
| 25% repair of defect depth | 1 |
| 0% repair of defect depth | 0 |
| Macroscopic appearance | |
| Intact smooth surface | 4 |
| Fibrillated surface | 3 |
| Small, scattered fissures or cracs | 2 |
| Several, small or few but large fissures | 1 |
| Total degeneration of grafted area | 0 |

### Macroscopic Evaluation

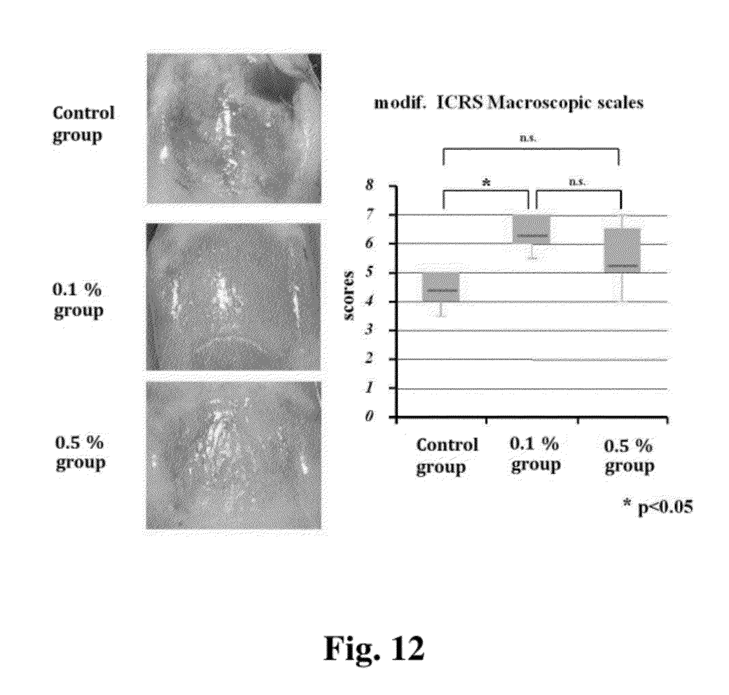

FIG. 12 shows images of knee joints of the respective groups collected as described above, and illustrates the results with a modified International Cartilage Repair Society (ICRS) score. Macroscopic observation revealed that the control group had many fissures on the surface, while the 0.1% (W/V) artificial collagen administration group had such a tendency that many knee joints have smooth surfaces and are similar to the surrounding cartilage with respect to the color tone. The 0.5% (W/V) artificial collagen administration group had many sparse fissures. Further, there was a significant difference between the 0.1% (W/V) artificial collagen administration group and the control group, and it could be confirmed that cartilage was repaired. As recognized from the result of modified ICRS score described in FIG. 12, cartilage regeneration could be achieved by the intraarticular administration of the artificial collagen aqueous solution as the substrate for cartilage cultivation of the present invention. It should be noted that the results with a modified ICRS score are expressed as an average of scores of 6 knees for the control group, an average of 6 knees for the 0.1% (W/V) group, and an average of 6 knees for the 0.5% (W/V) group. In addition, a Mann-Whitney U test was used for statistics.

### Safranin-0 Staining

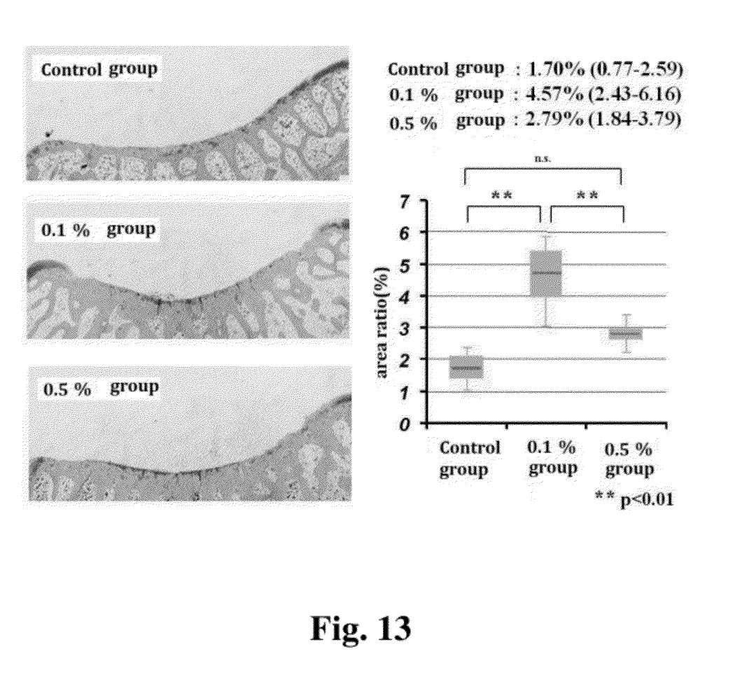

FIG. 13 shows images in which knee joints of the respective groups collected as described above have been stained with Safranin-0, and illustrates the results of a Safranin-0-stained area ratio. The stained areas of the 0.1% (W/V) artificial collagen administration group and the 0.5% (W/V) artificial collagen administration group were larger compared to the stained area of the control group. In particular, a proteoglycan was present in a large amount. In addition, the stained area ratios of the 0.1% (W/V) artificial collagen administration group and the 0.5% (W/V) artificial collagen administration group were larger compared to the stained area ratio of the control group. It should be noted that the portion stained with safranin-0 is a newly formed tissue, and is suggested to be the cartilage.

### Type 2 Collagen Staining

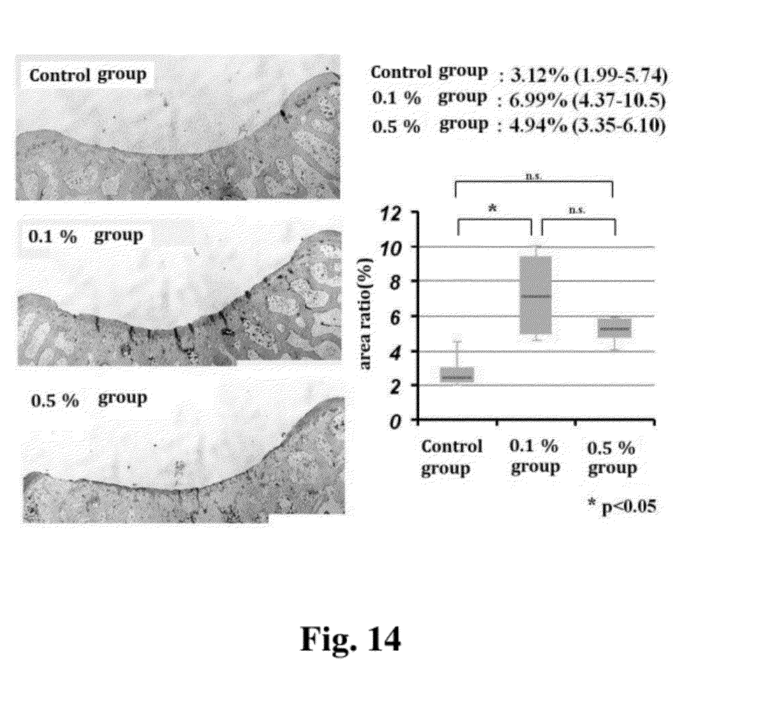

FIG. 14 shows images in which knee joints of the respective groups collected as described above have been immunostained with a type 2 collagen antibody, and illustrates the results of a type 2 collagen antibody-immunostained area ratio. The stained areas of the 0.1% (W/V) artificial collagen administration group and the 0.5% (W/V) artificial collagen administration group were larger compared to the stained area of the control group. In addition, the stained area ratios of the 0.1% (W/V) artificial collagen administration group and the 0.5% (W/V) artificial collagen administration group were larger compared to the stained area ratio of the control group. It should be noted that type 2 collagen is a major component which accounts for about 50% (V/V) of the cartilage dry weight, and hence, it is conceivable that the cartilage was regenerated in the stained portion.

### General Statement on Results of Example 3

Cartilage regeneration can be achieved by the intraarticular administration of the substrate for cartilage cultivation of the present invention in and around a cartilage defect site of a patient.

### EXAMPLE 4

### Confirmation of Action of Artificial Collagen Aqueous Solution of Present Invention on Cartilage Cells

Confirmation was made whether the artificial collagen aqueous solution of the present invention directly acted on cartilage cells. The details are as follows.

### Artificial Collagen

The artificial collagen used in the present invention is artificial collagen sold by PHG Corporation (INCI name: Poly(Tripeptide-6), CAS. No: 60961-94-6: www.phg.co.jp/research/collagen.html).

### Piece of Cartilage Used

A part (about 5×5×5 mm) of a piece of cartilage extirpated from a patient with knee osteoarthritis who underwent artificial knee joint surgery was used as a piece of cartilage.

### Organ Cultivation of Piece of Cartilage

Fluorescein isothiocyanate isomer-I (FITC)-labeled artificial collagen was added to a DMEM solution supplemented with 10% FBS so as to achieve a concentration of 0.5% (W/V), to thereby prepare a cultivation medium. The piece of cartilage was impregnated into the cultivation medium, and organ cultivation was performed under conditions at 37° C. for 72 hours (see Table 6 below). In addition, the piece of cartilage after organ cultivation was stained with safranin-0.

### Results of Organ Cultivation

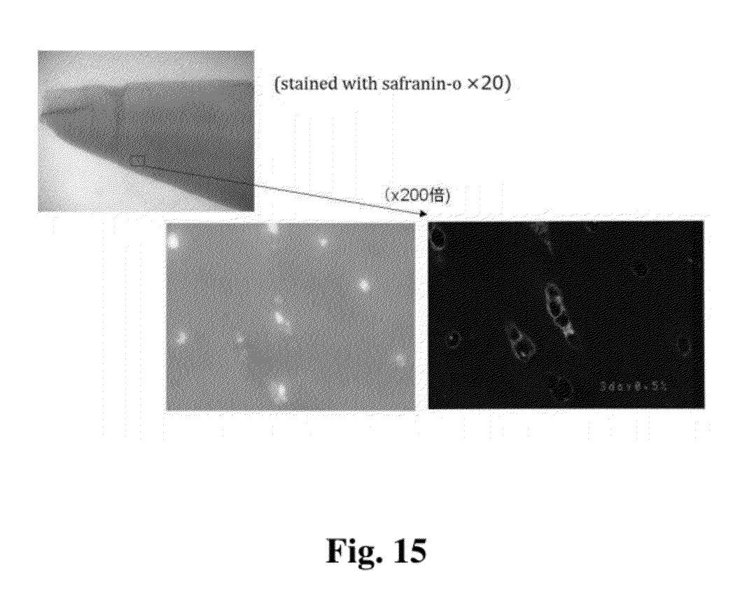

The piece of cartilage after the above-mentioned organ cultivation was observed with a fluorescence microscope (see FIG. 15). It was confirmed that FITC labels gathered around cartilage cells in the piece of cartilage, and the artificial collagen directly acted on cartilage cells.

### INDUSTRIAL APPLICABILITY

The substrate for cartilage cultivation of the present invention exhibits high safety and has a growth promoting effect on cartilage cells, and hence is very useful. In addition, cartilage regeneration can be achieved by the intraarticular administration of the substrate for cartilage cultivation of the present invention in and around a cartilage defect site.

1. A substrate for cartilage cultivation, comprising an artificial collagen.

2. The substrate for cartilage cultivation according to claim 1, wherein the artificial collagen comprises a solution having a concentration of 0.001 to 6.00% (W/V).

3. The substrate for cartilage cultivation according to claim 2, wherein the-solution is an aqueous solution.

4. The substrate for cartilage cultivation according to claim 1, wherein the artificial collagen comprises a polypeptide formed of peptide units represented by (1) to (3):
[—(OC—(CH₂)ₘ—CO)ₚ-(Pro-Y-Gly)ₙ-]ₐ; (1)
[—(OC—(CH₂)ₘ—CO)_{q}—(Z)ᵣ—]_{b}; (2)
and
[—HN—R—NH—]_{c}, (3)
wherein: m represents an integer of 1 to 18, p and q are identical to or different from each other and each represent 0 or 1, Y represents Pro or Hyp, and n represents an integer of 1 to 20; Z represents a peptide chain formed of 1 to 10 amino acid residues, r represents an integer of 1 to 20, and R represents a linear or branched alkylene group; and a ratio of a to b is a/b=100/0 to 30/70 (molar ratio), c=a is satisfied if p=1 and q=0, c=b is satisfied if p=0 and q=1, c=a+b is satisfied if p=1 and q=1, and c=0 is satisfied if p=0 and q=0.

5. The substrate for cartilage cultivation according to claim 4, wherein m represents an integer of 2 to 12, n represents an integer of 2 to 15, Z represents a peptide chain formed of at least one kind of amino acid residue or peptide residue selected from the group consisting of Gly, Sar, Ser, Glu, Asp, Lys, His, Ala, Val, Leu, Arg, Pro, Tyr, and Ile, r represents an integer of 1 to 10, and R represents a C₂ to C₁₂ alkylene group.

6. The substrate for cartilage cultivation according to claim 1, further including comprising sodium hyaluronate.

7. The substrate for cartilage cultivation according to claim 1, further including comprising an RGD peptide.

8. The substrate for cartilage cultivation according to claim 1, including further comprising a scaffold material for cartilage cells.

9. The substrate for cartilage cultivation according to claim 1, further comprising a material for differentiation and growth of cartilage cells.

10. A joint function improving agent, comprising sodium hyaluronate and artificial collagen.

11. The joint function improving agent according to claim 10, wherein the artificial collagen comprises an aqueous solution having a concentration of 0.001 to 6.00% (W/V).

12. The joint function improving agent according to claim 10, wherein the artificial collagen comprises a polypeptide formed of peptide units represented by (1) to (3):
[—(OC—(CH₂)ₘ—CO)ₚ-(Pro-Y-Gly)ₙ-]ₐ; (1)
[—(OC—(CH₂)ₘ—CO)_{q}—(Z)ᵣ—]_{b}; (2)
and
[—HN—R—NH—]_{c}, (3)
wherein: m represents an integer of 1 to 18, p and q are identical to or different from each other and each represent 0 or 1, Y represents Pro or Hyp, and n represents an integer of 1 to 20; Z represents a peptide chain formed of 1 to 10 amino acid residues, r represents an integer of 1 to 20, and R represents a linear or branched alkylene group; and a ratio of a to b is a/b=100/0 to 30/70 (molar ratio), c=a is satisfied if p=1 and q=0, c=b is satisfied if p=0 and q=1, c=a+b is satisfied if p=1 and q=1, and c=0 is satisfied if p=0 and q=0.

13. The joint function improving agent according to claim 12, wherein m represents an integer of 2 to 12, n represents an integer of 2 to 15, Z represents a peptide chain formed of at least one kind of amino acid residue or peptide residue selected from Gly, Sar, Ser, Glu, Asp, Lys, His, Ala, Val, Leu, Arg, Pro, Tyr, and Ile, r represents an integer of 1 to 10, and R represents a C₂ to C₁₂ alkylene group.

14.-17. (canceled)

18. A method of treatment for cartilage regeneration, comprising intra-articular injecting of the substrate for cartilage cultivation according to claim 1 in and around a site on a patient selected from the group consisting of a cartilage defect site and a cartilage disorder site.

19. A method of treatment for promotion of cartilage regeneration, comprising intra-articular injecting of the substrate for cartilage cultivation according to claim 1 in and around a site of a cartilage transplantation of a patient after the cartilage transplantation.

20. A method of treatment for protecting joint function, comprising intra-articular injecting of the substrate for cartilage cultivation according to claim 1 in and around a site on a patient suffering with joint damage selected from the group consisting of a cartilage defect site and a cartilage disorder.

21. The method of treatment for protecting joint function according to claim 20, wherein the joint damage is selected from the group consisting of:
(1) joint damage due to osteoarthritis;
(2) joint damage due to injury;
(3) joint damage due to sports;
(4) joint damage due to rheumatoid arthritis; and
(5) joint damage due to connective tissue disorder.

22. The method of treatment for protecting joint function according to claim 21, wherein the joint damage due to connective tissue disorder is joint damage due to systemic lupus erythematosus.
